(19) United States
(12) Patent Application Publication
(10) Pub. No.: US 2024/0269060 A1
(43) Pub. Date: Aug. 15, 2024

(54) THERMOSTABLE, POLYACRYLATE-FREE COSMETIC PREPARATION

(71) Applicant: BEIERSDORF AG, Hamburg (DE)

(72) Inventors: David SCHLENKER, Hamburg (DE); Christina JAPP, Hamburg (DE); Julia PRUNS, Hamburg (DE)

(21) Appl. No.: 18/564,721

(22) PCT Filed: May 4, 2022
(86) PCT No.: PCT/EP2022/061956

§ 371 Date: Nov. 28, 2023

(30) Foreign Application Priority Data
Jun. 4, 2021 (DE) 102021205683.0

(51) Int. Cl. A61K 8/92; A61K 8/35; A61K 8/37; A61K 8/73; A61Q 17/04

(52) U.S. Cl. CPC A61K 8/922; A61K 8/35; A61K 8/37; A61K 8/73; A61K 8/731; A61Q 17/04; A61K 2800/30

(57) ABSTRACT

Cosmetic preparation containing a) hydroxypropyl methylcellulose, b) xanthan gum and c) hydrogenated rapeseed oil which is free of glycerol tripalmitate, the preparation being free of basis of polyacrylates, carbomers and polyvinylpyrrolidones.

The present invention relates to a cosmetic preparation comprising hydroxypropyl methylcellulose, xanthan gum and hydrogenated rapeseed oil which is free of glycerol tripalmitate, the preparation being free of basis of polyacrylates, carbomers and polyvinylpyrrolidones.

For many years there has been an unbroken trend away from aristocratic pallor toward “healthy, athletically brown skin”. To achieve this, people expose their skin to solar radiation, since this causes pigment formation in the sense of melanin formation. However, the ultraviolet radiation of sunlight also has a harmful effect on the skin. Besides acute damage (sunburn), long-term damage such as an increased risk of developing skin cancer occurs in the case of excessive irradiation with light from the UVB range (wavelength: 280-320 nm). Excessive exposure to UVB and UVA radiation (wavelength: 320-400 nm) additionally leads to weakening of the elastic and collagen fibres in connective tissue. This leads to numerous phototoxic and photoallergic reactions, resulting in premature aging of the skin.

Therefore, to protect the skin, a range of photoprotective filter substances that can be used in cosmetic preparations have been developed. In most industrialized countries, these UVA and UVB filters are collated in the form of positive lists such as Annex 7 of the Kosmetikverordnung [German Cosmetics Ordinance].

The multiplicity of commercially available sunscreens must not, however, distract from the fact that these prior-art preparations have a range of disadvantages.

Cosmetic preparations provided in tube form must have a particular minimum viscosity so that they do not “leak out” of the tube, i.e. escape from the packaging provided with a screw cap when stored horizontally or “upside down”. This minimum viscosity is dependent on factors including the ambient temperature and usually decreases as the temperature increases.

One basic problem of cosmetic sunscreens in tube form is that this minimum viscosity has to be ensured even at relatively high ambient temperatures, since sunscreens are exposed to high ambient temperatures during transport and use outdoors, especially in the summer.

According to the prior art, polyacrylates and carbomers are used to ensure this minimum viscosity.

Polyacrylates are also used as a substitute for low molecular weight emulsifiers. Their emulsifying effect is based on the formation of a gel network in the preparation, which stabilizes the oil droplets in the aqueous environment and prevents them from coalescing.

The use of such polymers is viewed increasingly critically by the wider public. Concerns are often expressed in the public discussion that such polymers, analogous to so-called “microplastics”, can pose a burden on the environment. Whether such concerns are justified and scientifically valid can be left open in the context of the present disclosure. However, the fact is that cosmetics manufacturers and consumers are increasingly interested in developing alternative techniques without polyacrylates/carbomers. The preparations should be “microplastic-free” according to the consumers' understanding.

It was therefore the object of the present invention to develop a viscosity-stable cosmetic preparation and particularly a sunscreen in the case of which the use of polymeric film formers (particularly those based on polyacrylates and polyvinylpyrrolidones) can largely be dispensed with. The preparations should be suitable for being stored in tubes and being able to be applied from them.

Surprisingly, the objects are achieved by a cosmetic preparation comprising

a) hydroxypropyl methylcellulose,

b) xanthan gum and

c) hydrogenated rapeseed oil which is free of glycerol tripalmitate, the preparation being free of basis of polyacrylates, carbomers and polyvinylpyrrolidones.

The prior art does in principle include the use of hydrogenated rapeseed oil in such preparations, for example of the “Dermofeel Viscolid pof” hydrogenated rapeseed oil from Evonik. However, these hydrogenated rapeseed oils comprise glycerol tripalmitate. It was therefore not possible to obtain the inventive thermostability of the viscosity when using this raw material.

By contrast, one hydrogenated rapeseed oil that is particularly advantageous according to the invention is the “VGB22” hydrogenated rapeseed oil from Société Industrielle des Oléagineux. This is free of glycerol tripalmitate.

It is advantageous according to the invention if the preparation contains one or more UV filters selected from the group of the compounds 4-(tert-butyl)-4′-methoxydibenzoylmethane (INCI: Butyl Methoxydibenzoylmethane), hexyl 2-[4-(diethylamino)-2-hydroxybenzoyl]benzoate (INCI: Diethylamino Hydroxybenzoyl Hexyl Benzoate) and 2,4-bis {[4-(2-ethylhexyloxy)-2-hydroxy]phenyl}-6-(4-methoxyphenyl)-1,3,5-triazine (INCI: Bis-Ethylhexyloxyphenol Methoxyphenyl Triazine).

Embodiments of the present invention that are advantageous according to the invention are also characterized in that the preparation is free of 3-(4-methylbenzylidene)camphor, 2-hydroxy-4-methoxybenzophenone (INCI: Oxybenzone), 2-ethylhexyl 4-methoxycinnamate (INCI: Octyl Methoxycinnamate), parabens (particularly propyl and butyl paraben), methylisothiazolinone, chloromethylisothiazolinone and DMDM hydantoin, polyethylene glycol ethers or polyethylene glycol esters.

It is also advantageous in the sense of the present invention if the preparation is free of glycerol tripalmitate.

It is advantageous according to the invention if the preparation contains hydroxypropyl methylcellulose at a concentration of 0.05% to 0.5% by weight, based on the total weight of the preparation.

Furthermore, it is advantageous according to the invention if the preparation contains xanthan gum at a concentration of 0.1% to 1% by weight, based on the total weight of the preparation.

Last but not least, the embodiments of the present invention that are advantageous according to the invention are characterized in that the preparation contains the hydrogenated rapeseed oil c) (INCI: Hydrogenated Rapeseed Oil) at a concentration of 0.5% to 3% by weight, based on the total weight of the preparation.

Furthermore, it is advantageous according to the invention if the preparation contains one or more esters selected from the group of the compound isopropyl palmitate (INCI: Isopropyl Palmitate), di-n-butyl adipate (INCI: Butyl Adipate), butylene glycol dicaprylate/dicaprate (INCI: Butylene Glycol Dicaprylate/Dicaprate).

In such a case, for isopropyl palmitate the advantageous use concentration is 1% to 10% by weight, based on the total weight of the preparation.

In such a case, for di-n-butyl adipate the advantageous use concentration is 0.5% to 5% by weight, based on the total weight of the preparation.

In such a case, for butylene glycol dicaprylate/dicaprate the advantageous use concentration is 1% to 5% by weight, based on the total weight of the preparation.

Embodiments of the present invention that are advantageous according to the invention are also characterized in that the preparation contains carnauba wax.

In such a case, for carnauba wax the advantageous use concentration is 0.5% to 2% by weight, based on the total weight of the preparation.

It is advantageous according to the invention if the preparation is present in the form of an O/W emulsion.

If the preparation is in the form of an emulsion, then it is preferable according to the invention if the emulsifier used is Polyglyceryl-3 Methylglucose Distearate.

The preparation may otherwise contain the customary cosmetic ingredients and be composed like conventional preparations. That is to say that the preparations according to the invention contain the customary ingredients at the customary use concentrations.

One preparation that is advantageous according to the invention is last but not least characterized in that the preparation has a viscosity of 2500-3000 mPas⁻¹ after storage for 28 days at 40° C. This viscosity is determined according to the invention using the following method: Rheomat R123 from proRheo GmbH, spindle No. 1. Here, the device is first calibrated with the corresponding spindle according to the manufacturer's specifications. The viscosity is then measured in the prescribed vessel and the final value which is displayed after about 30 seconds is read off. The viscosity is measured on a preparation which has cooled to room temperature) (20° C.) after storage.

Also in accordance with the invention is an LDPE tube containing the cosmetic preparation according to the invention.

### EXAMPLES

The examples which follow are intended to illustrate the present invention without limiting it. Unless otherwise indicated. all amounts, proportions and percentages are based on the weight and the total amount or on the total weight of the preparations.

| | | | Specimen 3 |
|---|---|---|---|
| | Specimen 1 | Specimen 2 | (wax with |
| | (prior-art | (inventive | similar |
| | rapeseed oil) | rapeseed oil) | melting point) |
| INCI | m [%] | m [%] | m [%] |
| Isopropyl Palmitate | 6.00 | 6.00 | 6.00 |
| Aqua | 54.03 | 54.03 | 54.03 |
| Tocopheryl Acetate | 0.06 | 0.06 | 0.06 |
| Dibutyl Adipate | 3.00 | 3.00 | 3.00 |
| Aqua + Trisodium EDTA | 0.50 | 0.50 | 0.50 |
| Synthetic Beeswax | | | 2.00 |
| Butyl | 4.75 | 4.75 | 4.75 |
| Methoxydibenzoylmethane | | | |
| Phenylbenzimidazole | 0.50 | 0.50 | 0.50 |
| Sulfonic Acid | | | |
| Glycerin | 2.00 | 2.00 | 2.00 |
| Aqua + Sodium Hydroxide | 0.16 | 0.16 | 0.16 |
| Alcohol Denat. + Aqua | 7.00 | 7.00 | 7.00 |
| Copernicia Cerifera Cera | 1.50 | 1.50 | 1.50 |
| Ethylhexyl Salicylate | 2.00 | 2.00 | 2.00 |
| Xanthan Gum | 0.10 | 0.10 | 0.10 |
| Ethylhexyl Triazone | 4.75 | 4.75 | 4.75 |
| Ethylhexylglycerin | 0.15 | 0.15 | 0.15 |
| Polyglyceryl-3 | 1.00 | 1.00 | 1.00 |
| Methylglucose Distearate | | | |
| Bis-Ethylhexyloxyphenol | 3.50 | 3.50 | 3.50 |
| Methoxyphenyl Triazine | | | |
| Butylene Glycol | 2.00 | 2.00 | 2.00 |
| Dicaprylate/Dicaprate | | | |
| Microcrystalline | 1.00 | 1.00 | 1.00 |
| Cellulose + Cellulose Gum | | | |
| Diethylamino | 2.00 | 2.00 | 2.00 |
| Hydroxybenzoyl Hexyl | | | |
| Benzoate | | | |
| Hydrogenated Rapeseed | | 2.00 | |
| Oil**, free of glycerol | | | |
| tripalmitate | | | |
| C12-15 Alkyl Benzoate | 2.00 | 2.00 | 2.00 |
| Hydrogenated Rapeseed | 2.00 | | |
| Oil* (Rapeseed Oil) | | | |
| containing glycerol | | | |
| tripalmitate | | | |
| Total: | 100.00 | 100.00 | 100.00 |

### Results/Viscosities

The following viscosities were determined in accordance with the measurement method stated above:

| | Viscosity RT | Viscosity | Viscosity |
|---|---|---|---|
| INCI | t₀ | 40° C. 1 d | 40° C. 28 d |
| Rapeseed | 1600 mPas⁻¹ | 2350 mPas⁻¹ | 2450 mPas⁻¹ |
| Oil* | | | |
| Rapeseed | 1650 mPas⁻¹ | 2650 mPas⁻¹ | 2900 mPas⁻¹ |
| Oil** | | | |
| Synthetic | 1300 mPas⁻¹ | 1350 mPas⁻¹ | 1300 mPas⁻¹ |
| Beeswax | | | |

*= Dermofeel Viscolid pof hydrogenated rapeseed oil from Evonik
**= “VGB22” hydrogenated rapeseed oil from Société Industrielle des Oléagineux

1-12. (canceled)

13. A cosmetic preparation, wherein the preparation comprises
(a) hydroxypropyl methylcellulose,
(b) xanthan gum, and
(c) hydrogenated rapeseed oil which is free of glycerol tripalmitate, the preparation being free of polyacrylates, carbomers and polyvinylpyrrolidones.

14. The preparation of claim 13, wherein the preparation comprises one or more of 4-(tert-butyl)-4′-methoxydibenzoylmethane, hexyl 2-[4-(diethylamino)-2-hydroxybenzoyl]benzoate and 2,4-bis{[4-(2-ethylhexyloxy)-2-hydroxy]phenyl}-6-(4-methoxyphenyl)-1,3,5-triazine.

15. The preparation of claim 13, wherein the preparation is free of 3-(4-methylbenzylidene)camphor, 2-hydroxy-4-methoxybenzophenone, 2-ethylhexyl 4-methoxycinnamate, parabens, methylisothiazolinone, chloromethylisothiazolinone, DMDM hydantoin, polyethylene glycol ethers and polyethylene glycol esters.

16. The preparation of claim 13, wherein the preparation is free of glycerol tripalmitate.

17. The preparation of claim 13, wherein the preparation comprises from 0.05% to 0.5% by weight of (a), based on a total weight of the preparation.

18. The preparation of claim 13, wherein the preparation comprises from 0.1% to 1% by weight of (b), based on a total weight of the preparation.

19. The preparation of claim 17, wherein the preparation comprises from 0.1% to 1% by weight of (b), based on a total weight of the preparation.

20. The preparation of claim 13, wherein the preparation comprises from 0.5% to 3% by weight of (c), based on a total weight of the preparation.

21. The preparation of claim 18, wherein the preparation comprises from 0.5% to 3% by weight of (c), based on a total weight of the preparation.

22. The preparation of claim 19, wherein the preparation comprises from 0.5% to 3% by weight of (c), based on a total weight of the preparation.

23. The preparation of claim 13, wherein the preparation comprises one or more of isopropyl palmitate, di-n-butyl adipate, butylene glycol dicaprylate/dicaprate.

24. The preparation of claim 14, wherein the preparation comprises one or more of isopropyl palmitate, di-n-butyl adipate, butylene glycol dicaprylate/dicaprate.

25. The preparation of claim 23, wherein the preparation comprises from 1% to 10% by weight of isopropyl palmitate, based on a total weight of the preparation.

26. The preparation of claim 23, wherein the preparation comprises from 0.5% to 5% by weight of di-n-butyl adipate, based on a total weight of the preparation.

27. The preparation of claim 23, wherein the preparation comprises from 1% to 5% by weight of butylene glycol dicaprylate/dicaprate, based on a total weight of the preparation.

28. The preparation of claim 13, wherein the preparation comprises carnauba wax.

29. The preparation of claim 13, wherein the preparation is present as an O/W emulsion.

30. The preparation of claim 22, wherein the preparation is present as an O/W emulsion.

31. The preparation of claim 13. wherein the preparation has a viscosity of 2500-3000 mPas⁻¹ after storage for 28 days at 40° C.

32. The preparation of claim 13. wherein the preparation is present in a LDPE tube.
